(19) United States
(12) Patent Application Publication
(10) Pub. No.: US 2022/0390426 A1
(43) Pub. Date: Dec. 8, 2022

(54) AIE PROBE AND APPLICATION THEREOF

(71) Applicant: The Hong Kong University of Science and Technology, Hong Kong (CN)

(72) Inventors: Benzhong Tang, Hong Kong (CN); Yujie Tu, Hong Kong (CN)

(21) Appl. No.: 17/762,165

(22) PCT Filed: Mar. 2, 2021
(86) PCT No.: PCT/CN2021/078627

§ 371 Date: Mar. 21, 2022

Related U.S. Application Data
(60) Provisional application No. 63/100,195, filed on Mar. 2, 2020.

(51) Int. Cl. G01N 33/02; C09K 11/06; G01N 21/78; G01N 21/64

(52) U.S. Cl. CPC G01N 33/02; C09K 11/06; G01N 21/78; G01N 21/6428; C09K 2211/1007; C09K 2211/1018

(57) ABSTRACT

The present invention discloses an AIE probe exhibiting monotonic or nonmonotonic responses to pH change, aggregation-induced emission (AIE) characteristics. The present invention also discloses methods for detecting albumin protein and amine gas.

### CROSS REFERENCE TO RELATED APPLICATIONS

The present application is a continuation of International Application No. PCT/CN2021/078627, filed on Mar. 2, 2021, which this application also claims priority to and the benefit of U.S. Provisional Patent Application Ser. No. 63/100,195, filed on Mar. 2, 2020, the disclosure of which is incorporated by reference in its entirety.

### TECHNICAL FIELD

The present invention relates to compounds exhibiting monotonic or nonmonotonic responses to pH change, aggregation-induced emission (AIE) characteristics, and the use of the compounds.

### BACKGROUND

Human health and food safety must be considered when packaging and shipping raw food. Refrigeration processes have greatly increased the distances that raw food can travel by maintaining low temperatures to hinder the growth of microbes that lead to food spoilage. Many methods have been designed to monitor the shipping process, such as time-temperature indicators (TTI) to show whether raw food materials have been stored at elevated temperatures for an extended duration of time. However, maintaining a chilled temperature only slows down the enzymatic processes of food spoilage. Indeed, biogenic amine species, such as putrescine and cadaverine, produced by microbes have been found in rainbow trout stored at 0° C. by the second day.

Biogenic amines are good indicators of food freshness because they are products of microbial fermentation. In the process of food spoilage, microbes break down amino acids via deaminization to generate ammonia, and via decarboxylation to generate biogenic amines such as cadaverine, putrescine, spermidine, spermine, and others. These biogenic amines not only signal food spoilage, but also have adverse impact on human health and physiological functions. Thus, monitoring biogenic amines in food is important both because the chemical species can have toxic effects, and because they signify food spoilage by microbes. When compared to TTIs, which only respond to temperature changes, a system detecting the presence of biogenic amines offers a more direct method of monitoring food safety and hygiene.

The detection of biogenic amines can be achieved by exploiting their basic nature using probes or sensors that shows photophysical changes upon protonation/deprotonation. In fact, there are many molecular species which have visible color change (i.e., absorption change) upon protonation/deprotonation. Such a visual change allows for easy visualization of changes due to the presence of biogenic amines.

An ideal system for detection of biogenic amines would show not only absorption but also luminescence change. Luminescent systems can be much more sensitive to changes, resulting in significantly easier identification.

Fluorogens are widely used as probes or sensors to detect the local environmental changes, such as polarity, pH, temperature, viscosity, aggregation state, based on the stimulus-response (S/R) system. Usually, scientists aim to design the fluorescence system with monotonic S/R (e.g. increased stimulus→increased response). However, the more informative nonmonotonic S/R system (e.g. stimulus→response 1 then response 2) is seldom invented.

### SUMMARY

In order to overcome the drawbacks of prior arts, the present invention provides various embodiments described below.

In one embodiment, an AIE probe is provided. The AIE probe comprises a compound that exhibits aggregation induced emission properties, wherein the compound comprises a backbone structure:

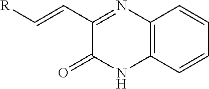

wherein R is selected from the group consisting of:

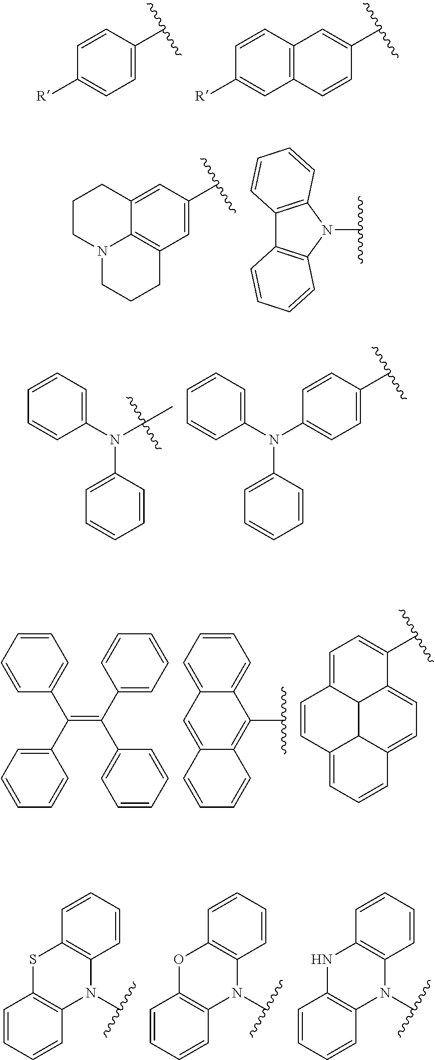

wherein R′ is selected from the group consisting of:

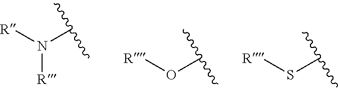

wherein R″, R″′, R″″, R′″″ are each independently selected from the group consisting of —H, —CH₃, and —CH₂CH₃.

In another embodiment, a method of detecting food spoilage in a sample is provided. The method comprises:

administering an AIE probe of the invention to the sample;

waiting for a period of time after administering the AIE probe; and

detecting the presence of food spoilage by measuring light emission.

In still another embodiment, a method of detecting food spoilage in a sample is provided. The method comprises:

administering an AIE probe of the invention to the packaged sample;

waiting for a first period of time, while gaseous amine generated from the spoilage sample, and the concentration of the gaseous amine increases with time and/or temperature, reaching a first threshold concentration;

detecting Level 1 of food spoilage by adapting the AIE probe change to a first color in response to the first threshold concentration;

waiting for a second period of time, while gaseous amine successively generated from the spoilage sample, reaching a second threshold concentration, greater than the first threshold concentration; and

detecting Level 2 of food spoilage by adapting the same AIE probe change to a second color, in response to the second threshold concentration.

The above description is only an outline of the technical schemes of the present invention. Preferred embodiments of the present invention are provided below in conjunction with the attached drawings to enable one with ordinary skill in the art to better understand said and other objectives, features and advantages of the present invention and to make the present invention accordingly.

### BRIEF DESCRIPTION OF THE DRAWINGS

The present invention can be more fully understood by reading the following detailed description of the preferred embodiments, with reference made to the accompanying drawings, wherein:

FIG. 1 The ¹H NMR spectrum of ASQ in DMSO-d⁶.

FIG. 2 The high-resolution mass spectrum of ASQ.

FIG. 3A PL spectra of ASQ in EtOH/water mixtures with different water fractions (f_{w}). FIG. 3B The plot of l/l₀ (red line) and wavelength (blue line) of emission peaks versus f_{w}. l₀ equals the intensity of f_{w}=0. [ASQ]=100 μM, λₑₓ=430 nm.

FIG. 4A The fluorescence response of ASQ to HSA. FIG. 4B Linear plot of l/l₀ at 542 nm versus HSA concentrations. l₀ equals to the intensity of [HSA]=0 mg/L. FIG. 4C Selectivity study of ASQ towards albumin. l₀ equals the intensity of the blank sample. [Biomolecules]=1 mg/mL. [ASQ]=100 μM, λₑₓ=430 nm.

FIG. 5A Photographs of color change towards protonation by addition of TFA in solution and fuming of TFA gas on filter papers. FIG. 5C Photographs of luminescent responses towards protonation by addition of TFA in solution and fuming of TFA gas on filter papers. FIG. 5B Absorption spectra of ASQ solution with the addition of TFA. FIG. 5D PL spectra of ASQ solution with the addition of TFA. Equivalence of TFA/ASQ=0˜200 and 1500˜65000.

FIG. 6A The dual fluorescence changes of ASQ-2H⁺ upon ammonia gas fuming. FIG. 6B Demonstration of food spoilage detection of salmon meat (top) and small dissected fish (bottom) over 24 h.

### DETAILED DESCRIPTION

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

In the luminescence researches, twisted intramolecular charge transfer (TICT) is a very common solvent effect in systems with electron donor-acceptor structures, featured by the large red-shifted and weakened emission as the solvent polarity increases. If both the donor and acceptor are nitrogen-containing moieties, they are anticipated to be protonation-responsive. Protonation on the donor can weaken the D-A interactions to result in a blue-shifted emission, while protonation on the acceptor can strengthen the D-A interaction, thus giving a red-shifted emission. Therefore, by assembling a protonatable acceptor and donor, the resulting molecule exhibits a nonmonotonic color (wavelength, λ) response to pH change. This is a main design strategy of this invention. Furthermore, to expand this idea, molecules with more complex structure to have proton load/unload acceptor and donor are also provided.

In a preferred embodiment of this invention, we designed aggregation-induced emission luminogens (AIEgen) exhibiting nonmonotonic responses to pH change, and used one of them, namely, 4-(dimethylamino)styryl)quinoxalin-2(1H)-one (ASQ) as an example to prove the feasibility in the detection of albumin protein and amine gas. In this invention, “AIEgen,” “luminogen,” “AIE probe,” “AIE sensor,” “probe,” “sensor,” and “fluorogen” are used interchangeably.

Detection of albumin protein: The detection of blood and urine albumin is clinically significant to examine health status and monitor chronic kidney diseases. However, the present instrument or immunoassay-based techniques are expensive and time-consuming. Since the fluorescence method is advantageous in cost and time efficiency, sensitivity, specificity, etc., different fluorogens for albumin detection were invented majorly based on two design approaches:

(1) Fluorogens with aggregation-induced emission (AIE) characteristics are sensitive to environmental constrain. They are weakly emissive in free state but emissive after binding with albumin due to the restriction of molecular motion mechanism (e.g. U.S. 20130177991A1).

(2) Fluorogens with twistied intramolecular charge transfer (TICT) effect are sensitive to environmental polarity change. They are weakly emissive in polar aqeous solution but emissive after binding in nonpolar cavities of albumin (e.g CN105838355A).

In the present invention, the probes which exihibit both AIE and TICT properties are promising in specific and quantitative analysis of albumin in biological fluids and serves as a fluorescent assay for cheap and fast detection in-time and on-site. Moreover, the AIEgen—albumin hybrid nanocomposites as biocompatible materials have been increasingly used in drug delivery, bioimaging, photothermal therapy, etc. Thus, the probes could be promising choices in different albumin-related applications.

Detection of amine gas: biogenic amines as the products of microbial fermentation, are good indicators of food spoilage, especially for seafood. In order to achieve fast and real-time monitoring of food freshness, the optical methods (i.e. absorption, fluorescence) are good options that are more straightforward, sensitive, and visible than other analytical methods such as gas chromatography. Primarily, the probes should be pH-sensitive, displaying absorption/fluorescence changes upon protonation/deprotonation. Meanwhile, they should be AIE-active since they would be used in the solid state. In previous intentions (e.g. WO2018210272A1), the fluorescence of the probe can be turned on upon amine exposure (brightness1→brightness2). However, in the present invention, the fluorescence of the probe shows either turn-on effect or ratiometric color change (color1→color2). Particularly, if the probe has two protonatable sites on the electron donor and acceptor moieties (e.g. ASQ), the optical property change could be nonmonotonic (e.g. colon color2→color3). Therefore, the probes in this invention with AIE-active+pH-sensitive properties and different modes of fluorescence response are ideal for different pH/amine gas detection related applications including food safety monitoring.

In a first embodiment of the present invention, an AIE probe is provided. The AIE probe comprising a compound that exhibits aggregation induced emission properties, wherein the compound comprises a backbone structure:

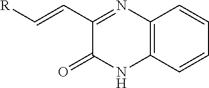

wherein R is selected from the group consisting of:

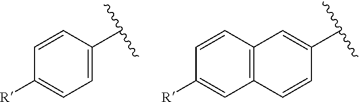

wherein R′ is

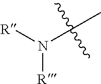

wherein R″ and R″′ are each independently selected from the group consisting of —H, —CH₃₆, and —CH₂CH₃.

In this embodiment, the unsubstituted imine nitrogen on the heterocyclic electron acceptor moiety and the nitrogen on the R electron donor moiety can be protonated. Furthermore, the protonated imine nitrogen can also be deprotonated.

In a second embodiment of the present invention, an AIE probe is provided. The AIE probe comprising a compound that exhibits aggregation induced emission properties, wherein the compound comprises a backbone structure:

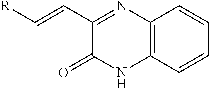

wherein R is selected from the group consisting of:

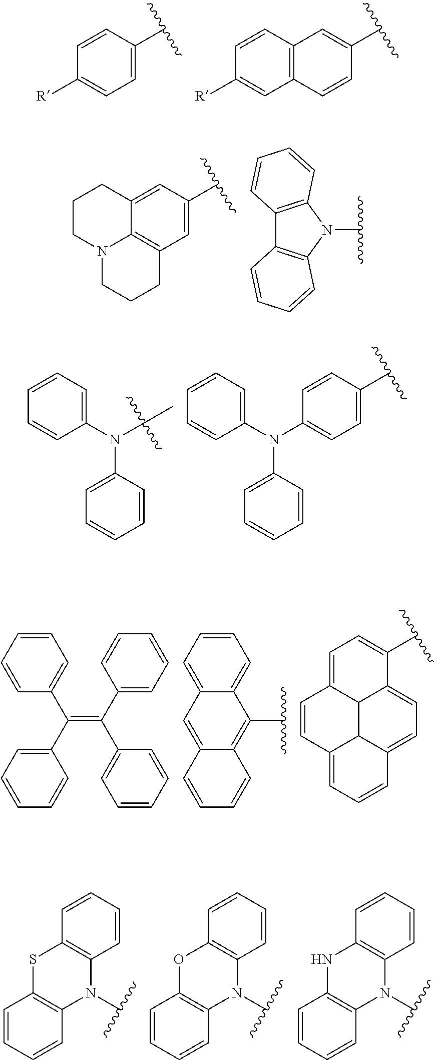

wherein R′ is selected from the group consisting of:

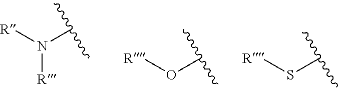

wherein R″, R″′, R″″, R′″″ are each independently selected from the group consisting of —H, —CH₃, and —CH₂CH₃.

In this embodiment, the heterocyclic electron acceptor moiety and the R electron donor moiety can be protonated (or proton loaded). Furthermore, the protonated heterocyclic electron acceptor moiety and the R electron donor moiety can also be deprotonated (or proton unloaded). In this invention, “protonated” and “proton loaded” are used interchangeably; “deprotonated” and “proton unloaded” are used interchangeably.

In this embodiment, protonation on the heterocyclic electron acceptor moiety give rise to a detectable red shift in light absorption and light emission. Protonation on the R electron donor moiety gives rise to a detectable blue shift in light absorption and light emission.

Deprotonation of the heterocyclic electron acceptor moiety give rise to a detectable blue shift in light absorption and light emission. Deprotonation of the R electron donor moiety gives rise to a detectable red shift in light absorption and light emission.

In one example of this embodiment, the AIE probe exhibits aggregation induced emission upon exposure to an amine, e.g. gaseous amine.

In a third embodiment, a method of detecting food spoilage in a sample is provided. The method comprising:

administering an AIE probe of the invention to the sample;

waiting for a period of time after administering the AIE probe; and

detecting the presence of food spoilage by measuring light emission.

In this embodiment, the AIE probe is loaded onto a solid substrate (e.g. filter paper strip) prior to administering the AIE probe to the sample.

Moreover, the light emission is an absorption color change and/or fluorescence in response to UV excitation.

In one example of this embodiment, further comprising determining food safety by observing absorption color change and/or fluorescence color change.

In a fourth embodiment, a method of detecting food spoilage in a sample is provided. The method comprising:

administering an AIE probe of the invention to the packaged sample;

waiting for a first period of time, while gaseous amine generated from the spoilage sample, and the concentration of the gaseous amine increases with time and/or temperature, reaching a first threshold concentration;

detecting Level 1 of food spoilage by adapting the AIE probe change to a first color in response to the first threshold concentration;

waiting for a second period of time, while gaseous amine successively generated from the spoilage sample, reaching a second threshold concentration, greater than the first threshold concentration; and

detecting Level 2 of food spoilage by adapting the same AIE probe change to a second color, in response to the second threshold concentration.

In this embodiment, the AIE probe is loaded onto a solid substrate (e.g. filter paper strip) prior to administering the AIE probe to the sample.

Additionally, the second color is distinct from the first color.

Moreover, “change to a first color” is an absorption color change and/or fluorescence in response to UV excitation; “change to a second color” is an absorption color change and/or fluorescence in response to UV excitation.

In one example of this embodiment, further comprising determining food safety by observing absorption color change and/or fluorescence color change.

In a fifth embodiment, a kit for monitoring food safety is provided. The kit comprises: an AIE probe of this invention; a solid substrate, wherein the AIE probe is loaded onto the solid substrate (e.g. filter paper strip); and a packaged food product.

In a sixth embodiment, a luminescent hybrid nanocomposite is provided, comprising an AIE probe of this invention and albumin.

The following examples are provided to illustrate further and to facilitate the understanding of the present teachings and are not in any way intended to limit the invention.

### EXAMPLES

### Example 1—Synthesis of Dihydroquinoxaline Derivatives

ASQ were synthesized by a condensation reaction between 3-methylquinoxalin-2(1H)-one and 4-(dimethylamino)benzaldehyde derivatives; An exemplary reaction scheme and process are provided below:

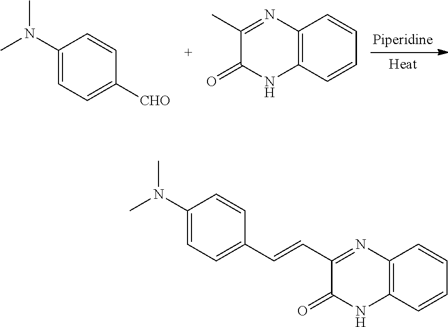 [SMILES: CN(C)c1ccc(C=O)cc1 ; Cc1nc2ccccc2[nH]c1=O ; CN(C)c1ccc(/C=C/c2nc3ccccc3[nH]c2=O)cc1]

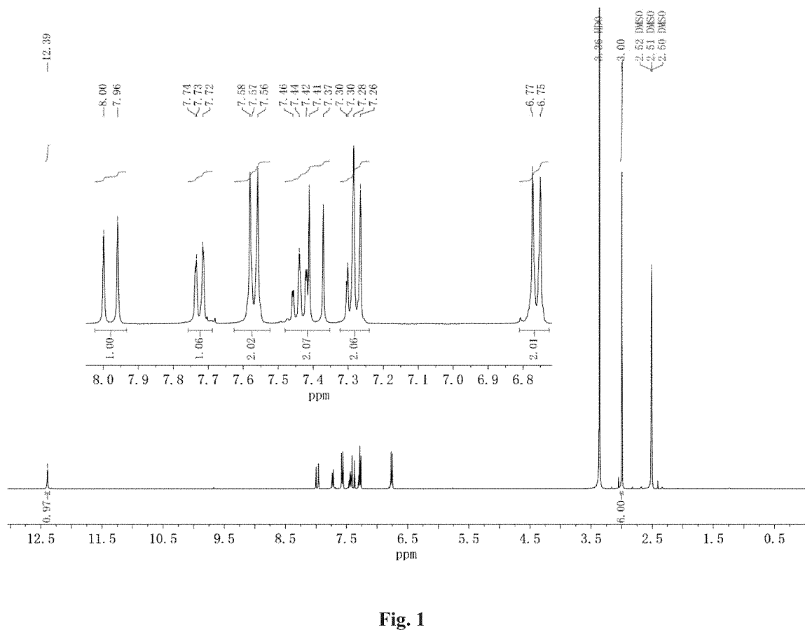

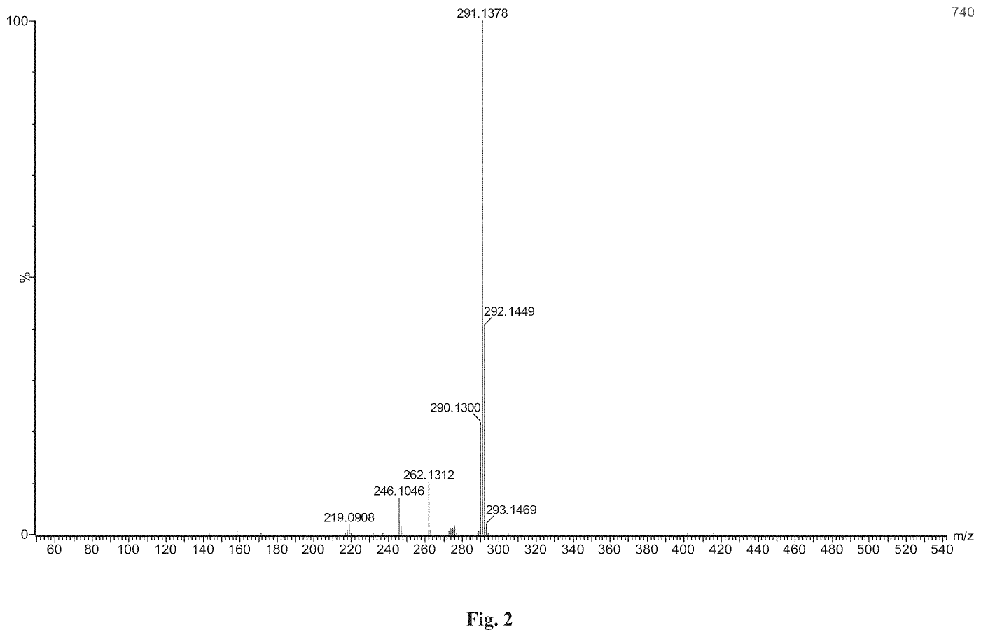

1.60 g of 3-methylquinoxalin-2(1H)-one and 1.49 g of 4-(dimethylamino)benzaldehyde were mixed in a round-bottom flask and heated up to 160° C., then 5 ml piperidine was added into the mixture. It was observed that the pale-yellow mixture turned red rapidly. After 10 min, 50 of mL of ethanol was added into the resulting mixture. Then the suspension was filtrated and washed by ethanol (15 mL×3). The washed product was further purified by the column chromatography. The chemical structures and purity of ASQ were confirmed by standard spectroscopic techniques including nuclear magnetic resonance (NMR) and high-resolution mass spectrometry (HRMS) (FIG. 1 and FIG. 2).

### Example 2—AIE Property of ASQ

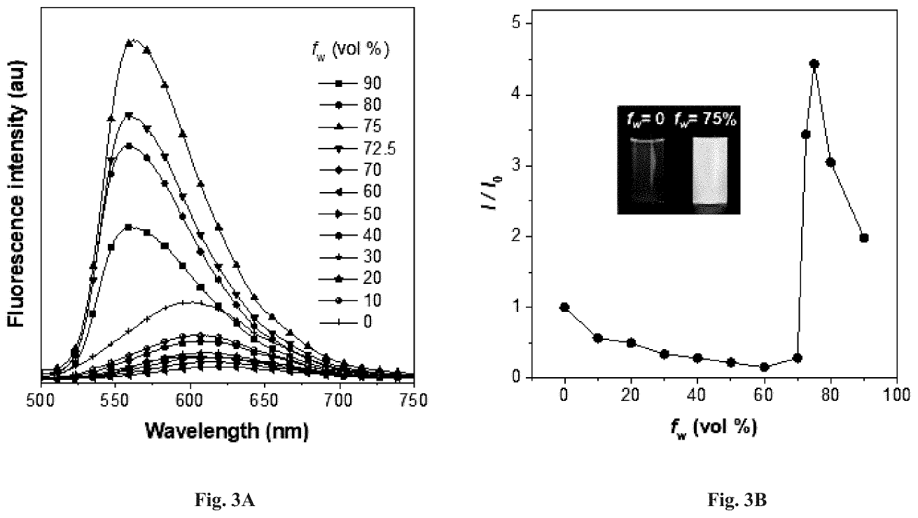

The AIE property of ASQ was studied in the ethanol/water mixture. Once water as a poor solvent was added into the ASQ's ethanol solution, the emission will firstly decrease and red-shifted due to the increased polarity (FIG. 3B, f_{w}=0˜60%). As aggregate forms, the emission is enhanced (FIG. 3B, f_{w}=60%-75%).

### Example 3—Albumin Detection of ASQ

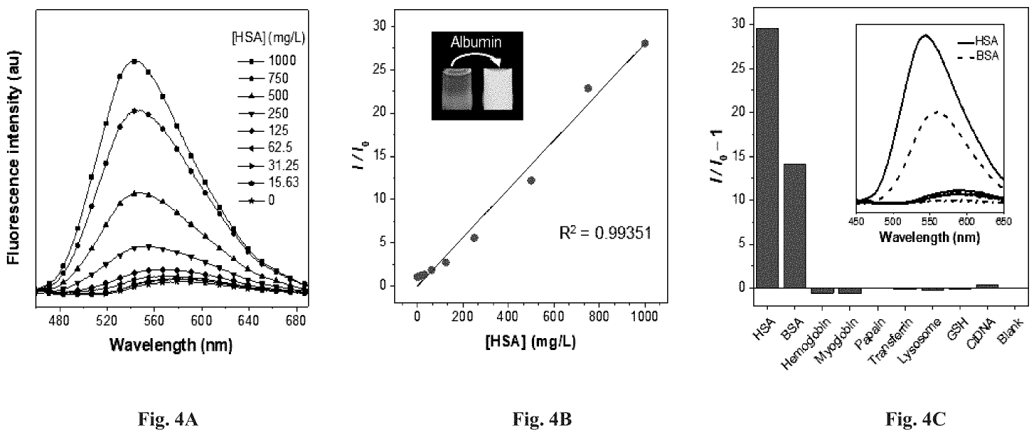

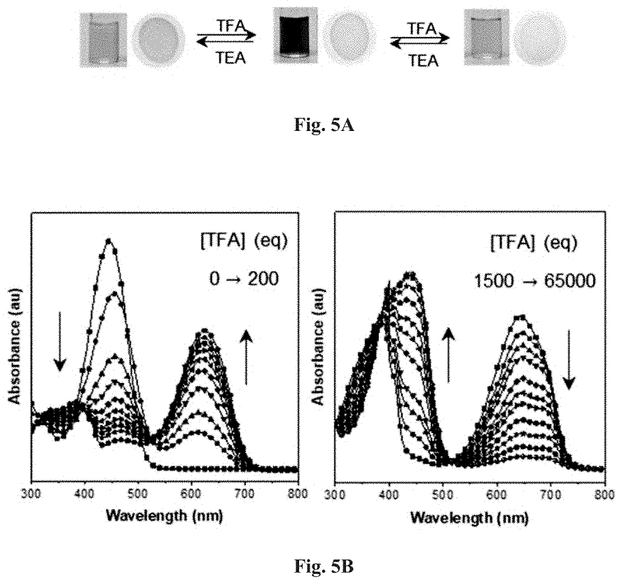

Albumin protein is the substance carrier in blood with multiple polar or nonpolar binding sites. ASQ was found to be a suitable substrate of albumin. When albumin was added in the ASQ's PBS buffer solution, the emission can be greatly enhanced (FIG. 4A). There is a very good linear relationship between the fluorescence intensity and albumin concentration (FIG. 4B). Meanwhile, ASQ has a good selectivity towards albumin since its emission cannot be turned on by binding with other common biomolecules such as hemoglobin (FIG. 4C).

### Example 4—Response to Protonation/pH of ASQ

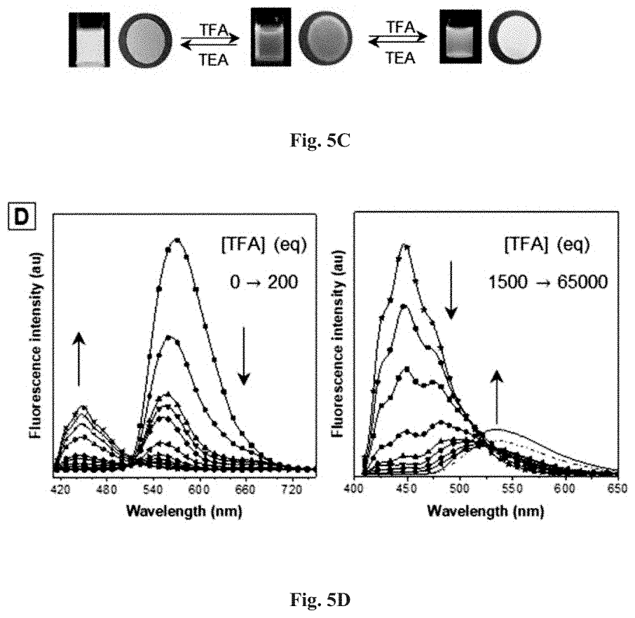

Either in the solution state or the solid state, ASQ shows exactly two appearance color and fluorescence color change upon the continuous addition of trifluoroacetic acid (TFA) or TFA gas fuming (FIG. 5A/ FIG. 5C). The process is reversed by the addition of triethylamine (TEA) or TEA gas fuming. FIGS. 5B and 5D show the absorption and fluorescence changes during the sequential protonation process. The DCM solution of ASQ shows an absorption peak at 445 nm (λ_{ab}@445 nm) and a fluorescence peak at 570 nm (λ_{f}@570 nm). When the [TFA]/[ASQ] ratio increases from 0 to around 200, the λ_{ab}@445 nm gradually drops while the λ_{ab}@643 nm rises up. The λ_{f}@570 nm also declines but the λ_{f}@447 nm intensifies. When the [TFA]/[ASQ] ratio further increases from 1500 to 65000, the λ_{ab}@643 nm goes down and the λ_{ab}@435 nm increases. It seems that the absorption spectrum is gradually switched back to the original pattern. The fluorescence spectrum undergoes a similar “backward” shift that the λ_{f}@447 nm declines and the λ_{f}@512 nm increases. In summary, upon decreasing pH, the fluorescence blue-shifts and then red-shifts, thus a nonmonotonic responses to protonation stimuli.

### Example 5—Biogenic Amine Gas Detection of ASQ

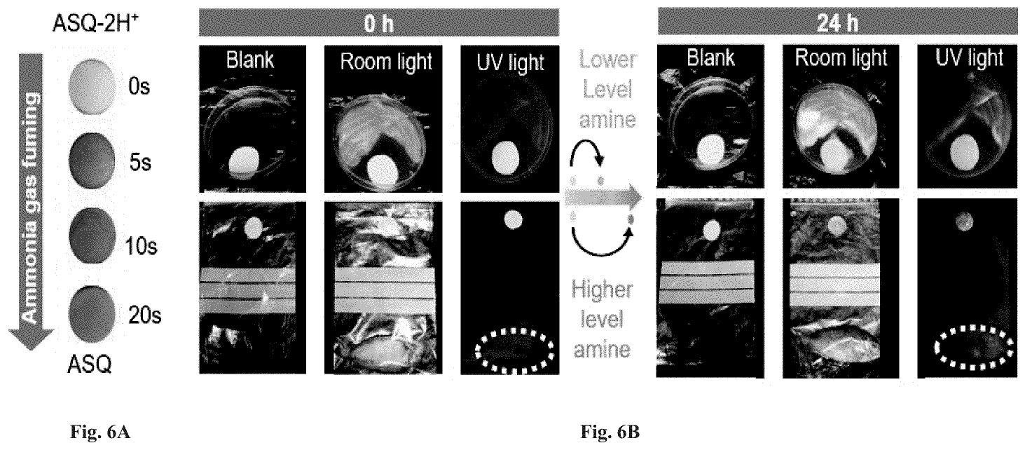

ASQ can serve as a sensor for volatile basic gas such as biogenic amine gas. The deterioration of protein gives rise to smelly amine species which are indicators of food spoilage. The pre-acidified ASQ-2H⁺ has a yellow appearance and emits yellow fluorescence. When a test paper stained by ASQ-2H⁺ is exposed under the atmosphere of ammonia, ASQ-2H⁺ starts to be gradually deprotonated to be ASQ-H⁺ with the blue appearance and blue fluorescence, then ASQ with the orange appearance and orange fluorescence (FIG. 6A). The two trends of nonmonotonic changes can help people distinguish the freshness of perishable food in an easy and straightforward way. We used the eatable salmon meat and eviscerated dead fish as a demonstration. After 24 h storage under the same condition, the test paper in the salmon meat package turned to be blue but the test paper in dead fish package turned to be orange, which means the amine generated by the dead fish is at a higher level (FIG. 6B). Therefore, unlike the previous reports that only one trend of color or intensity change was observed, the ASQ system can not only tell us whether the food goes bad but also tell us how severe the food spoilage is by the distinct color change gradient. In this scenario, the nonmonotonic S/R system can certainly do more than the normal monotonic S/R system.

The above embodiments are only used to illustrate the principles of the present invention, and they should not be construed as to limit the present invention in any way. The above embodiments can be modified by those with ordinary skill in the art without departing from the scope of the present invention as defined in the following appended claims.

What is claimed is:

1. An AIE probe comprising a compound that exhibits aggregation induced emission properties, wherein the compound comprises a backbone structure: 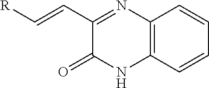
wherein R is selected from the group consisting of: 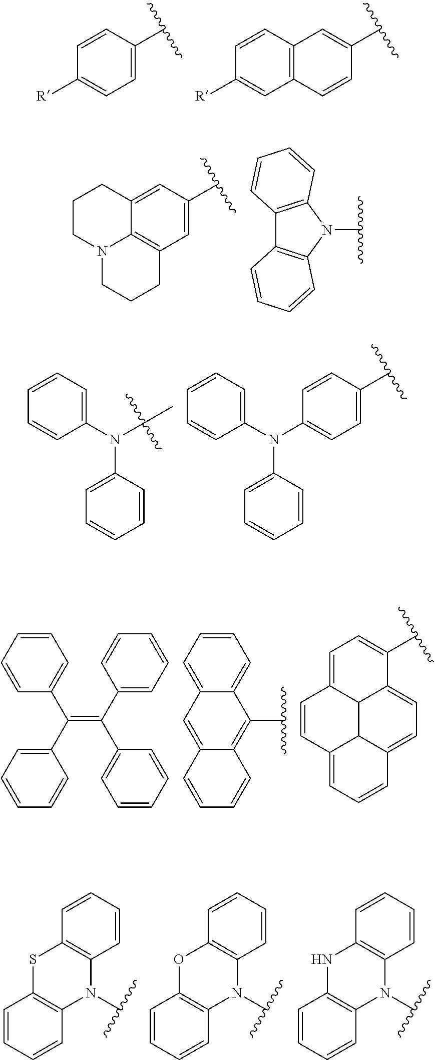
wherein R′ is selected from the group consisting of: 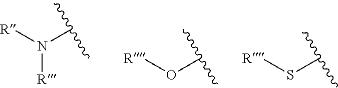
wherein R″, R″′, R″″, R′″″ are each independently selected from the group consisting of —H, —CH₃, and —CH₂CH₃.

2. The AIE probe of claim 1, wherein the compound comprises a backbone structure: 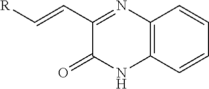
wherein R is selected from the group consisting of: 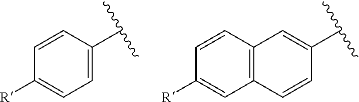
wherein R′ is 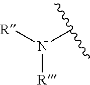
wherein R″ and R″′ are each independently selected from the group consisting of —H, —CH₃, and —CH₂CH₃.

3. The AIE probe of claim 2, wherein the unsubstituted imine nitrogen on the heterocyclic electron acceptor moiety and the nitrogen on the R electron donor moiety can be protonated.

4. The AIE probe of claim 1, wherein the heterocyclic electron acceptor moiety and the R electron donor moiety can be protonated.

5. The AIE probe of claim 4, wherein protonation on the heterocyclic electron acceptor moiety give rise to a detectable red shift in light absorption and light emission.

6. The AIE probe of claim 4, wherein protonation on the R electron donor moiety gives rise to a detectable blue shift in light absorption and light emission.

7. The AIE probe of claim 4, wherein deprotonation of the heterocyclic electron acceptor moiety give rise to a detectable blue shift in light absorption and light emission.

8. The AIE probe of claim 4, wherein deprotonation of the R electron donor moiety gives rise to a detectable red shift in light absorption and light emission.

9. The AIE probe of claim 1, wherein the AIE probe exhibits aggregation induced emission upon exposure to an amine.

10. The AIE probe of claim 9, wherein the amine is a gaseous amine.

11. A method of detecting food spoilage in a sample, comprising:
administering the AIE probe of claim 1 to the sample;
waiting for a period of time after administering the AIE probe; and
detecting the presence of food spoilage by measuring light emission.

12. The method of claim 11, wherein the AIE probe is loaded onto a solid substrate prior to administering the AIE probe to the sample.

13. The method of claim 11, wherein the light emission is an absorption color change.

14. The method of claim 11, wherein the light emission is fluorescence in response to UV excitation.

15. The method of claim 11, further comprising determining food safety by observing absorption color change and/or fluorescence color change.

16. A method of detecting food spoilage in a packaged sample, comprising:
administering the AIE probe of claim 1 to the packaged sample;
waiting for a first period of time, while gaseous amine generated from the spoilage sample, and the concentration of the gaseous amine increases with time and/or temperature, reaching a first threshold concentration;
detecting Level 1 of food spoilage by adapting the AIE probe of claim 1 change to a first color in response to the first threshold concentration;
waiting for a second period of time, while gaseous amine successively generated from the spoilage sample, reaching a second threshold concentration, greater than the first threshold concentration; and
detecting Level 2 of food spoilage by adapting the same AIE probe change to a second color, in response to the second threshold concentration.

17. The method of claim 16, wherein the AIE probe is loaded onto a solid substrate prior to administering the AIE probe to the sample.

18. The method of claim 16, wherein the second color is distinct from the first color.

19. A kit for monitoring food safety, comprising:
an AIE probe of claim 1;
a solid substrate, wherein the AIE probe is loaded onto the solid substrate; and
a packaged food product.

20. A luminescent hybrid nanocomposite, comprising an AIE probe of claim 1 and albumin.
